(19) United States
(12) Patent Application Publication
(10) Pub. No.: US 2017/0108512 A1
(43) Pub. Date: Apr. 20, 2017

(54) USE OF BIOMARKERS FOR DETECTION OF EXCESSIVE ALCOHOL USE

(71) Applicant: Indiana University Research and Technology Corporation, Indianapolis, IN (US)

(72) Inventor: Suthat Liangpunsakul, Carmel, IN (US)

(21) Appl. No.: 15/285,828

(22) Filed: Oct. 5, 2016

Related U.S. Application Data
(60) Provisional application No. 62/242,639, filed on Oct. 16, 2015.

(51) Int. Cl. G01N 33/68

(52) U.S. Cl. CPC G01N 33/6893; G01N 2800/307; G01N 2333/70596; G01N 2333/70578

(57) ABSTRACT

Methods of diagnosing excessive alcohol use are disclosed herein. More particularly, the present disclosure is directed to methods of diagnosing excessive alcohol use by identifying expression levels of various serum biomarkers. In some embodiments, expression levels of these biomarkers are affected by alcohol use even up to 30 days after consumption of alcohol.

### CROSS REFERENCE TO RELATED APPLICATIONS

This application claims priority under 35 USC §119(e) to U.S. Provisional Application Ser. No. 62/242,639 filed on Oct. 19, 2015, the entire disclosure of which is incorporated herein by reference.

### STATEMENT OF GOVERNMENT SUPPORT

This invention was made with government support under W81XWH-12-1-0497 awarded by U.S. Army Medical Research & Material Command and 1101CX000361-01A1 merit award by the Veterans Administration. The government has certain rights in the invention.

### BACKGROUND OF THE DISCLOSURE

The present disclosure relates generally to diagnosing excessive alcohol use. More particularly, the present disclosure is directed to diagnosing excessive alcohol use by identifying expression levels of various serum biomarkers. In some embodiments, expression levels of these biomarkers are affected by alcohol use even up to 30 days after consumption of alcohol.

Alcoholism is the most costly health problem in many countries. The cost, for example, in America is estimated to be about $117,000,000,000 per year. The treatment methods currently used are not very effective. Most alcoholics drop out of treatment within a month or two. Few alcoholics, regardless of the type of treatment, are able to avoid relapses and renewed alcohol abuse.

At present, laboratory markers to detect recent excessive alcohol consumption are limited by marginal sensitivity and specificity, uncertain interval of detection, and/or considerable cost. Direct measurement of alcohol concentration in blood and urine samples is not useful as it is only present for a short time after drinking cessation, and thus does not provide information more than a few hours beyond the most recent period of alcohol use. The plasma levels of enzymes expressed in the liver, such as gamma-glutamyl transpeptidase (GGT), aspartate aminotransferase (AST), alanine aminotransferase (ALT), and the mean corpuscular volume (MCV) of erythrocytes, are among the commonly used markers to identify chronic alcohol use. However, in a recent study, the ability of these markers to determine the levels of alcohol consumption in the preceding month revealed low sensitivities. Although percentage of carbohydrate-deficient transferrin (% CDT), a form of the serum iron carrying protein transferrin with altered carbohydrate composition, is a more specific marker for identifying chronic excessive alcohol use and monitoring abstinence, it does not have the desired sensitivity and specificity. Substantial efforts have been made to construct interview formats that correctly identify those with alcohol use disorder (AUD), such as Alcohol Use Disorders Identification Test Consumption (AUDIT-C; Hawkins et al., 2010), CAGE (Skogen et al., 2011), or reports from collateral family who interact with the subject (Whitford et al., 2009). Limitations of this approach are most apparent in cases where individuals are motivated to deny or minimize the magnitude of drinking behavior to mitigate personal ramifications.

Analysis of serum proteins offers a promising approach to quantifiable estimation of recent excessive alcohol consumption. Immoderate drinking has been shown to affect several organ systems, which may be reflected in changes in quantity or quality of constituent or novel plasma proteins or protein fragments. Organ- and/or tissue-specific proteins may be released into the blood stream when cells are injured by alcohol, or when systemic changes are induced by alcohol. In addition, because ethanol (EtOH) metabolism generates the highly reactive protein modifying reagent acetaldehyde, acetaldehyde-protein adducts have been identified in many laboratories (Conduah Birt et al., 1998; Niemela, 1999; Roy et al., 2002).

The general approach to serum- and plasma-based biomarker development has been to remove the most abundant proteins to improve the resolution of low-abundance protein candidates. However, it has become clear after several years of pursuing this strategy that many of the very low-abundance peptide and protein markers are either present in undetectable levels or absent as a result of being excreted by the urinary system or bound to high-abundance carrier proteins in the plasma (Nomura et al., 2004; Stibler et al., 1979).

The current methods for treating alcoholism are not very successful probably because they do not effectively weaken the alcoholic's alcohol-drinking response. Some methods (e.g., counseling, support groups (e.g., Alcoholics Anonymous)) are aimed at increasing the alcoholic's ability or will power to withstand the drive for alcohol. The drive, however, is not weakened and the patient is told that he will remain an alcoholic, that is, a person with an overly strong alcohol-drinking response, for the rest of his life. These methods succeed in some alcoholics, but in most, the time eventually comes when a momentary decrease in will power causes a resumption of alcohol drinking and alcohol abuse.

Accordingly, there exists a need to diagnose excessive alcohol use many hours after drinking has ceased. Particularly, if the sensitivity and specificity of serum markers to identify subjects with excessive recent alcohol use could be improved, then clinical care would be enhanced. The sensitive biomarkers would confirm self-report of alcohol consumption, but also provide results from an objective biochemical test to help physicians motivate patients to moderate or stop drinking, and would provide objective measures of progress toward that goal.

### BRIEF DESCRIPTION OF THE DISCLOSURE

Because the low molecular weight portion of the human blood proteome is comprised of peptides and protein fragments, it has attracted significant interest, yet remains relatively unmined. Binding to high-abundance carrier proteins such as albumin prevents excretion by the kidneys, extending the half-lives of these potential disease biomarkers (also referred to herein as “markers”). A blood sample preparation approach that takes advantage of this inherent biological enrichment of disease-associated low molecular weight biomarkers has been coupled with high-resolution mass spectrometry and discriminant pattern analysis. In the present disclosure, this approach was used to identify markers for excessive alcohol use. It is believed that the serum proteome in excessive alcohol use subjects qualitatively and quantitatively differs from the proteome of those who drink only in moderation.

The present disclosure employs a proteomic method from serum samples and has identified proteins that appear to provide improved performance in detection of patients with excessive alcohol use, compared to conventional measures.

In one particular aspect, the present disclosure is related to a method for diagnosing excessive alcohol use in a subject. The method comprises: obtaining a sample from a subject suspected of excessive alcohol use; contacting the sample with an agent that specifically binds to a biomarker selected from the group consisting of AT-Rich Interactive Domain-Containing Protein 4B (ARID4B), phosphatidylcholine-sterol acyltransferase (LCAT), hepatocyte growth factor-like protein (MST1), ADP-ribosylation factor 6 (ARL6), sCD14, and sCD163, and combinations thereof, to form a complex between the agent and biomarker; detecting the complex to determine an expression level of the biomarker in the sample; and diagnosing excessive alcohol use in the subject if the expression level of the biomarker is increased as compared to the expression level of the biomarker in a reference sample.

In another aspect, the present disclosure is directed to a method for diagnosing excessive alcohol use in a subject. The method comprises: obtaining a sample from a subject suspected of excessive alcohol use; analyzing the sample by liquid chromatography/mass spectrometry to determine a concentration of a biomarker selected from the group consisting of AT-Rich Interactive Domain-Containing Protein 4B (ARID4B), phosphatidylcholine-sterol acyltransferase (LCAT), hepatocyte growth factor-like protein (MST1), ADP-ribosylation factor 6 (ARL6), sCD14, and sCD163, and combinations thereof; and diagnosing excessive alcohol use in the subject if the concentration level of the biomarker is increased as compared to the concentration of the biomarker in a reference sample.

In yet another aspect, the present disclosure is directed to a method for diagnosing excessive alcohol use in a subject. The method comprises: obtaining a sample from a subject suspected of excessive alcohol use; contacting the sample with an agent that specifically binds to sCD40, to form a complex between the agent and sCD40; detecting the complex to determine an expression level of sCD40 in the sample; and diagnosing excessive alcohol use in the subject if the expression level of the sCD40 is decreased as compared to the expression level of the sCD40 in a reference sample.

In yet another aspect, the present disclosure is directed to a biomarker panel for diagnosing excessive alcohol use, the biomarker panel comprising at least two biomarkers selected from the group consisting of AT-rich interactive domain-containing protein 4B (ARID4B), ETS domain-containing transcription factor ERF (ERF), actin-like protein 6A (ACTL6A), IgG (immunoglobulin lambda), phosphatidylcholine-sterol acyltransferase (LCAT), intercellular adhesion molecule 2 (ICAM2), hepatocyte growth factor-like protein (MST1), and ADP-ribosylation factor 6 (ARL6), sCD14, sCD163, and sCD40.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts a schematic diagram of the study design used in Example 1.

FIGS. 2A-2D depict proteomic analyses of serum from controls and excessive drinkers as analyzed in Example 1. FIG. 2A depicts the top 10 list of proteins stratified by their function from 602 unique proteins which were identified from serum. FIG. 2B depicts 47 and 4 proteins that were up- and down-regulated, respectively, in serum of subjects with excessive drinkers compared to controls. FIG. 2C depicts the function of 51 proteins which were found to be significantly different between excessive drinkers and controls. FIG. 2D depicts the fold-change of proteins in serum of excessive drinkers versus controls. The fold changes ratio >1.8 (upregulated) were selected as the cutoff values to designate significant changes in the protein expression which were validated with enzyme-linked immunosorbent assays.

FIGS. 3A-3H depict the abundance of the 8 candidate proteins which levels were at least 1.8-fold higher in subjects with excessive alcohol use compared to controls; identified from LC/MS: AT-rich interactive domain-containing protein 4B (ARID4B) (FIG. 3A), ETS domain-containing transcription factor ERF (ERF) (FIG. 3B), actin-like protein 6A (ACTL6A) (FIG. 3C), IgG (immunoglobulin lambda) (FIG. 3D), phosphatidylcholine-sterol acyltransferase (LCAT) (FIG. 3E), intercellular adhesion molecule 2 (ICAM2) (FIG. 3F), hepatocyte growth factor-like protein (MST1) (FIG. 3G), and ADP-ribosylation factor 6 (ARL6) (FIG. 3H). *p<0.05, when compared to controls.

FIGS. 4A-4D depicts dot plots demonstrating the levels of serum protein based on the enzyme-linked immunosorbent assays from the serum of excessive drinkers and controls in the verification cohort.

FIGS. 5A-5I depict receiver operating characteristics for performance of the tests to diagnose excessive drinkers in verification cohort.

FIGS. 6A-6I depict the relationship between marker of interest and the level of alcohol consumption in the last 30 days.

FIGS. 7A-7H depict the relationship between conventional markers, serum LPS, sCD14, and sCD163 and the amount of alcohol consumption during the last 30 days using TLFB as analyzed in Example 2. The data showed no linear relationship between AST (FIG. 7A), GGT (FIG. 7B), ALT (FIG. 7C), and MCV (FIG. 7D) and the number of drinks during the past month. Though there was a linear trend for % CDT (FIG. 7E) and total drinks, the r was only 0.38. A good association between the markers of monocyte activation and total drinks during the last month was observed (FIGS. 7F-7H).

FIG. 8 depicts levels of sCD40 in controls and EAU subjects, *p<0.05, as analyzed in Example 3.

FIG. 9 depicts levels of neopterin in controls and EAU subjects, *p<0.05, as analyzed in Example 4.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

As used herein, the methods are directed to be used with a subject susceptible to or at risk of a specified disease, disorder, or condition. More particularly, the methods of the present disclosure are to be used with a subset of subjects who are susceptible to or at elevated risk of excessive alcohol use (EAU). Subjects may be susceptible to or at elevated risk for excessive alcohol use due to family history, age, environment, and/or lifestyle. Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

As used herein, “susceptible” and “at risk” refer to having little resistance to a certain disease, disorder or condition, including being genetically predisposed, having a family history of, and/or having symptoms of the disease, disorder or condition.

As used herein, “excessive alcohol use” is defined by the National Institutes of Health/National Institute on Alcohol Abuse and Alcoholism and refers to a male subject having one or more of the following characteristics: (1) subject who consumes greater than 4 standard drinks in a day; and (2) subject who consumes greater than 14 standard drinks per week. Further, for a female subject, “excessive alcohol use” refers to female subjects having one or more of the following characteristics: (1) subject who consumes greater than 3 standard drinks in a day; and (2) subject who consumes greater than 7 standard drinks per week.

As used herein, “expression level of a biomarker” refers to the process by which a gene product is synthesized from a gene encoding the biomarker as known by those skilled in the art. The gene product can be, for example, RNA (ribonucleic acid) and protein. Expression level can be quantitatively measured (i.e., amount/concentration of biomarker) by methods known by those skilled in the art such as, for example, northern blotting, amplification, polymerase chain reaction, microarray analysis, tag-based technologies (e.g., serial analysis of gene expression and next generation sequencing such as whole transcriptome shotgun sequencing or RNA-Seq), Western blotting, enzyme linked immunosorbent assay (ELISA), liquid chromatography mass spectrometry (LC/MS), and combinations thereof.

As used herein, “a reference expression level of a biomarker” refers to the expression level of a biomarker established for a subject who does not engage in excessive alcohol use as determined by one skilled in the art using established methods as described herein, and/or a known expression level of a biomarker obtained from literature. The reference expression level of the biomarker can also refer to the expression level of the biomarker established for any combination of subjects such as a subject who does not consume alcohol, a subject who consumes moderate levels of alcohol, and expression level of the biomarker for a subject who has not consumed alcohol within the last 30 days prior to the time the sample is obtained from the subject, but who later consumes alcohol. The reference expression level of the biomarker can also refer to the expression level of the biomarker obtained from the subject to which the method is applied. As such, the change within a subject from visit to visit can indicate an increased or decreased risk for excessive alcohol use. For example, a plurality of expression levels of a biomarker can be obtained from a plurality of samples obtained from the same subject and used to identify differences between the pluralities of expression levels in each sample. Thus, in some embodiments, two or more samples obtained from the same subject can provide an expression level(s) of a blood biomarker and a reference expression level(s) of the blood biomarker.

Exemplary reference expression levels for various biomarkers discussed herein include those shown in the Table below:

| | | | EAU (excessive | | |
|---|---|---|---|---|---|
| Biomarker | Controls | Range for controls | alcohol use) | Range for EAU | p-value |
| sCD14 (ng/ml) | 1,266 ± 448 | 692.2-2,648.2 | 3,353 ± 1444 | 1,581.9-6,980.4 | 0.001 |
| sCd163 (ng/ml) | 85 ± 8.7 | 72-108 | 396.9 ± 171 | 111-753 | 0.001 |
| sCD40 (pg/ml) | 978.7 ± 412.1 | 354.2-1785.7 | 347.6 ± 108.5 | 134.1-606.5 | 0.0001 |
| Urinary neopterin | 160.4 ± 80.5 | 37.6-287.6 | 379.3 ± 85.5 | 221.93-543.42 | 0.0001 |
| (μmol/mol creatinine) | | | | | |

In general, for determining the respective expression levels allowing to establish the desired diagnosis in accordance with the respective embodiment of the present disclosure, (“threshold”, “reference amount”), the expression levels of the respective biomarkers are determined in appropriate patient groups. According to the diagnosis to be established, the patient group may, for example, comprise only healthy subjects (i.e., subjects who do not consume alcohol or who consume moderate levels of alcohol), or may comprise healthy subjects and subjects suffering from the pathophysiological state which is to be determined (i.e., excessive alcohol use), or may comprise only subjects suffering from the pathophysiological state which is to be determined, by the respective marker(s) using validated analytical methods. The results which are obtained are collected and analyzed by statistical methods known to the person skilled in the art. The obtained threshold values are then established in accordance with the desired probability of suffering from the condition and linked to the particular threshold value. For example, it may be useful to choose the median value, the 60th, 70th, 80th 90th, 95th or even the 99th percentile of the healthy and/or non-healthy patient collective, in order to establish the threshold value(s) or reference value(s).

A reference expression level of a diagnostic marker can be established, and the amount of the marker in a patient sample can be compared to the reference value. The sensitivity and specificity of a diagnostic and/or prognostic test depends on more than just the analytical “quality” of the test-they also depend on the definition of what constitutes an abnormal result. In practice, Receiver Operating Characteristic curves, or “ROC” curves, are typically calculated by plotting the value of a variable versus its relative frequency in “normal” and “disease” populations. For any particular marker of the disclosure, a distribution of marker expression levels or concentrations for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create a ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al, Radiology 143: 29-36 (1982).

In certain embodiments, markers and/or marker panels are selected to exhibit at least 70% sensitivity, in some cases at least 80% sensitivity, or even at least 85% sensitivity, at least 90% sensitivity, and in some cases at least 95% sensitivity, combined with at least 70% specificity, at least 80% specificity, at least 85% specificity, at least 90% specificity, and at least 95% specificity. In exemplary embodiments, both the sensitivity and specificity are at least 75% and in some cases at least 80%, at least 85%, at least 90%, and even at least 95%.

The present disclosure generally relates to carrier-bound proteins that show significant alterations in subjects with excessive alcohol use when compared to controls. These proteins are involved in inflammatory responses, cellular organization, enzymatic processes, protein transportation, and cell proliferation. More particularly, these proteins include AT-rich interactive domain-containing protein 4B (ARID4B), ETS domain-containing transcription factor ERF (ERF), actin-like protein 6A (ACTL6A), IgG (immunoglobulin lambda), phosphatidylcholine-sterol acyltransferase (LCAT), intercellular adhesion molecule 2 (ICAM2), hepatocyte growth factor-like protein (MST1), and ADP-ribosylation factor 6 (ARL6), sCD14, sCD163, sCD40, neopterin, and combinations thereof. Other suitable proteins are described in Liangpunsakul et al., “Novel Serum Biomarkers for Detection of Excessive Alcohol Use,” Alocholism: Clinical and Experimental Research, vol. 39, no. 3, March, 2015, which is incorporated herein by reference to the extent it is consistent herewith.

These newly identified proteins are mechanistically linked to alcohol consumption and metabolism. Particularly, AT-Rich Interactive Domain-Containing Protein 4B (ARID4B), encoded by the ARID4B gene, is a subunit of the histone deacetylase-dependent SIN3A transcriptional corepressor complex. This protein possesses several cellular function including proliferation, differentiation, and apoptosis.

Phosphatidylcholine-sterol acyltransferase (LCAT) is an enzyme that catalyzes the reaction between phosphatidylcholine and sterol, participating in glycerophospholipid metabolism and normally secreted from the liver into circulation. This functionality is in accordance with previous findings of alterations in the enzyme's activity and an increase in serum phospholipids in EtOH-fed mice, compared to controls, and that several of the associated lipids are under the control of LCAT.

Hepatocyte growth factor-like protein (MST1) is an inflammatory cytokine and can activate macrophages.

ADP-ribosylation factor 6 (ARL6) is a member of ADP-ribosylation factor family of GTP-binding proteins. It is a major regulator of vesicle biogenesis in intra-cellular traffic. Cells with increased ADP-ribosylation factor-like protein were found to have increased protein phosphatase 2A activity.

sCD14, sCD163 and sCD40 are proteins encoded by cluster of differentiation (CD) genes 14, 163 and 40, respectively. CD14 acts as a co-receptor for the detection of bacterial lipopolysaccharide (LPS, and can bind LPS only in the presence of lipopolysaccharide-binding protein (LBP). CD163 is a scavenger receptor for the hemoglobin-haptoglobin complex. It has also been shown to mark cells of monocyte/macrophage lineage. The soluble form of the receptor exists in plasma and is generated by ectodomain shedding of the membrane bound receptor. sCD163 is upregulated in a large range of inflammatory diseases including liver cirrhosis, type 2 diabetes, macrophage activation syndrome, Gaucher's disease, sepsis, HIV infection, rheumatoid arthritis and Hodgkin Lymphoma. CD40 is a costimulatory protein found on antigen presenting cells and is required for their activation. The protein receptor encoded by this gene is a member of the TNF-receptor superfamily. This receptor has been found to be essential in mediating a broad variety of immune and inflammatory responses including T cell-dependent immunoglobulin class switching, memory B cell development, and germinal center formation.

Neopterin (6-D-erythro-trihydroxypropylpterin) is a byproduct of tetrahydrobiopterin (BH4) synthesis. Increased amounts of neopterin are found to be produced by human monocytes/macrophages upon stimulation with the cytokine interferon-y. Therefore, measurement of neopterin concentrations in body fluids like serum, cerebrospinal fluid or urine provides information about activation of T helper cell 1 derived cellular immune activation. Increased neopterin production further has been previously found in infections by viruses including human immunodeficiency virus (HIV), infections by intracellular living bacteria and parasites, autoimmune diseases, malignant tumor diseases and in allograft rejection episodes, as well as in neurological and cardiovascular diseases cellular.

Based on these findings, the present disclosure is directed to diagnosing excessive alcohol use in a subject suspected of excessive alcohol use. According to the methods of the present disclosure, increased amounts of biomarker proteins or a variant thereof in comparison to reference amounts measured in a sample, for example a serum sample of a subject are indicative for excessive alcohol use. Other suitable samples may include urine, plasma, and whole blood samples. More particularly, increased amounts of any one or more of: AT-Rich Interactive Domain-Containing Protein 4B (ARID4B), phosphatidylcholine-sterol acyltransferase (LCAT), hepatocyte growth factor-like protein (MST1), ADP-ribosylation factor 6 (ARL6), sCD14, sCD163, and neopterin in comparison to reference amounts measured in a sample of a subject are indicative for excessive alcohol use.

In another embodiment, decreased amounts of a particular biomarker protein, such as sCD40, or a variant thereof in comparison to reference amounts measured in a sample, for example a serum sample of a subject, are indicative for excessive alcohol use.

In the context of the present disclosure, the biomarker amounts may be determined at least 6 hours after consumption of alcohol, including at least 12 hours after consumption of alcohol, including at least 24 hours after consumption of alcohol, including at least 48 hours after consumption of alcohol, at least one week after consumption of alcohol, including at least two weeks after consumption of alcohol, and even including at least 30 days after consumption of alcohol.

In accordance with the present disclosure, determining the amount of a biomarker can be achieved by all known suitable methods for determining the amount of a peptide or polypeptide in a sample. Said methods comprise immunoassay devices and methods which may utilize labelled molecules in various sandwich, competition, or other assay formats. Said assays will develop a signal which is indicative for the presence or absence of the peptide or polypeptide. Moreover, the signal strength can be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. Said methods comprise, for example, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays, cobalt-binding assay (CBA), and latex agglutination assays.

According to the instant disclosure, determining the amount of a biomarker peptide or polypeptide may comprise the steps of (a) contacting the biomarker with a specific ligand, (optionally) removing non-bound ligand, (b) measuring the amount of bound ligand. The bound ligand will generate an intensity signal. Binding according to the present disclosure includes both covalent and non-covalent binding. A ligand according to the present disclosure can be any compound, e.g., a peptide, polypeptide, nucleic acid, or small molecule, binding to the peptide or polypeptide described herein. Exemplary ligands include antibodies, nucleic acids, peptides or polypeptides such as receptors or binding partners for the peptide or polypeptide and fragments thereof comprising the binding domains for the peptides, and aptamers, e.g. nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g. phage display. Antibodies as referred to herein include both polyclonal and monoclonal antibodies, as well as fragments thereof, such as Fv, Fab and F(ab)2 fragments that are capable of binding antigen or hapten. The present disclosure also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present disclosure means that the ligand or agent should not bind substantially to (“cross-react” with) another peptide, polypeptide or substance present in the sample to be analyzed. The specifically bound peptide or polypeptide should be bound with at least 3 times higher, and in some embodiments will be bound with at least 10 times higher and even at least 50 times higher affinity than any other relevant peptide or polypeptide. Non-specific binding may be tolerable, if it can still be distinguished and measured unequivocally, e.g. according to its size on a Western Blot, or by its relatively higher abundance in the sample. Binding of the ligand can be measured by any method known in the art. Said method will be semi-quantitative or quantitative. Suitable methods are described in the following.

According to the instant disclosure, the term “antibody” refers to an antibody binding to a peptide selected from the group consisting AT-Rich Interactive Domain-Containing Protein 4B (ARID4B) or a variant thereof, phosphatidylcholine-sterol acyltransferase (LCAT) or a variant thereof, hepatocyte growth factor-like protein (MST1) or a variant thereof, ADP-ribosylation factor 6 (ARL6) or a variant thereof, sCD14 or a variant thereof, sCD163 or a variant thereof, sCD40 or a variant thereof, and neopterin or a variant thereof.

First, binding of a ligand may be measured directly, e.g. by NMR or surface plasmon resonance.

Second, if the ligand also serves as a substrate of an enzymatic activity of the peptide or polypeptide of interest, an enzymatic reaction product may be measured (e.g. the amount of a protease can be measured by measuring the amount of cleaved substrate, e.g. on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, the amount of substrate may be saturating. The substrate may also be labeled with a detectable label prior to the reaction. In some embodiments, the sample is contacted with the substrate for an adequate period of time. An adequate period of time refers to the time necessary for a detectable, (and in some cases measurable), amount of product to be produced. Instead of measuring the amount of product, the time necessary for appearance of a given (e.g. detectable) amount of product can be measured.

Third, the ligand may be coupled covalently or non-covalently to a label allowing detection and measurement of the ligand. Labelling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag may be at the N-terminus and/or C-terminus Suitable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star™ (Amersham Biosciences), ECF™ (Amersham Biosciences). A suitable enzyme-substrate combination may result in a colored reaction product, fluorescence or chemoluminescence, which can be measured according to methods known in the art (e.g. using a light-sensitive film or a suitable camera system). As for measuring the enzymatic reaction, the criteria given above apply analogously. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. Typical radioactive labels include 35s, 1251, 32p, ³³P and the like. A radioactive label can be detected by any method known and appropriate, e.g. a light-sensitive film or a phosphor imager. Suitable measurement methods according the present disclosure also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamid gel electrophoresis (SDS-PAGE), Western Blotting, and mass spectrometry), can be used alone or in combination with labeling or other detection methods as described above.

The amount of a biomarker peptide or polypeptide may be determined as follows: (a) contacting a solid support comprising a ligand for the peptide or polypeptide as specified above with a sample comprising the peptide or polypeptide and (b) measuring the amount peptide or polypeptide which is bound to the support. The ligand, which may be chosen from the group consisting of nucleic acids, peptides, polypeptides, antibodies and aptamers, may be present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the disclosure. Suitable methods for fixing/immobilizing said ligand are well known and include, but are not limited to ionic, hydrophobic, covalent interactions and the like. It is also contemplated to use “suspension arrays” as arrays according to the present disclosure (Nolan 2002, Trends Biotechnol. 20(1):9-12). In such suspension arrays, the carrier, e.g. a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The term “amount” as used herein encompasses the absolute amount of a biomarker polypeptide or peptide, the relative amount or concentration of the said polypeptide or peptide as well as any value or parameter which correlates thereto or can be derived there from. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “comparing” as used herein encompasses comparing the amount of the peptide or polypeptide comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample or a ratio of amounts is compared to a reference ratio of amounts.

### EXAMPLES

### Example 1

In this Example, serum biomarkers were used to identify subjects with excessive recent alcohol use.

### Materials and Methods

Quantitative proteomics based on label-free quantitative mass spectrometry on serum samples obtained from a discovery cohort was utilized. The validity of the findings was then tested in a verification cohort of similar composition and the results were compared to an assay of the enzymes currently used for detection of excessive drinking.

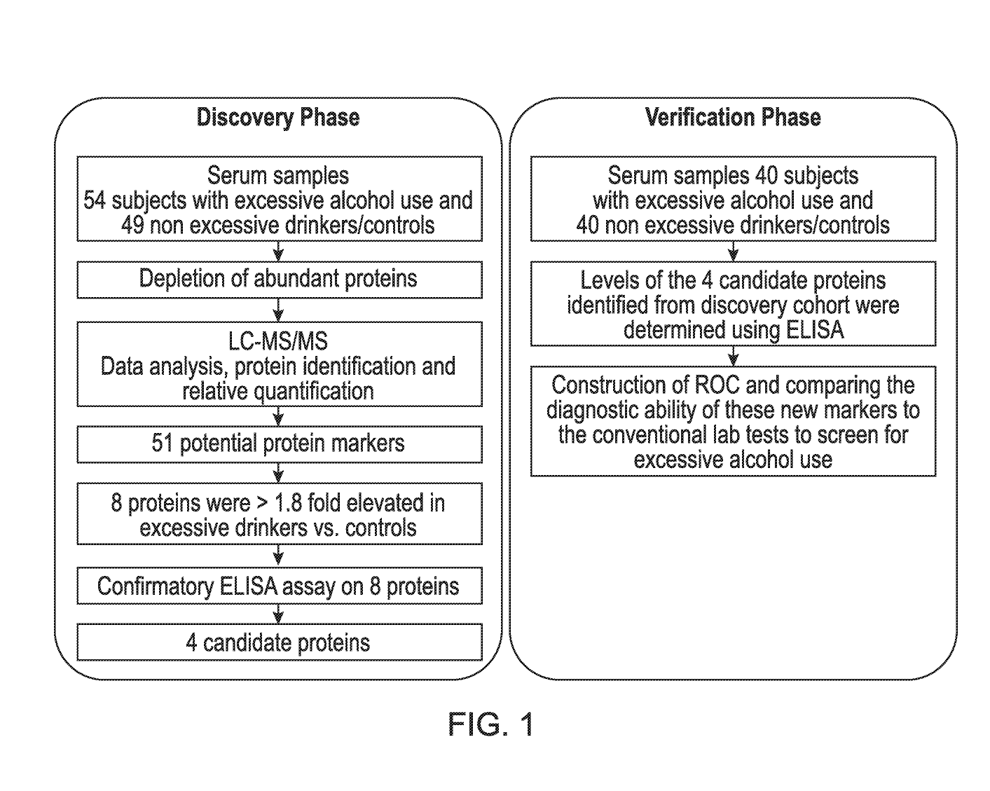

The outline of the methodology is shown in FIG. 1. For the discovery cohort, 54 subjects with a history of alcohol use disorder (AUD), who were admitted for alcohol rehabilitation at Fairbanks Drug and Alcohol Treatment Center (Indianapolis, Ind.), were recruited. They all met the criteria for AUD (defined by the DSM IV criteria) and “excessive drinking,” the latter defined by the National Institutes of Health/National Institute on Alcohol Abuse and Alcoholism as men who drink more than 4 standard drinks in a day (or more than 14 per week) and women who drink more than 3 standard drinks in a day (or more than 7 per week). Forty-nine non-excessive drinkers were recruited from Roudebush Veterans Administration Medical Center (RVAMC) in Indianapolis, Ind. The verification cohort comprised 40 subjects with AUD (from Fairbanks) and 40 nonexcessive drinker controls (from RVAMC). The inclusion criteria required subjects to be at least 21 years of age or older and be able to provide informed consent. Subjects were excluded if they had active and serious medical diseases (such as congestive heart failure, chronic obstructive pulmonary disease, cancer, uncontrolled diabetes, and chronic renal failure) at the time of screening, had history of any systemic infection within 4 weeks prior to the study, or had history of recent major surgeries within the past 3 months. The study design and protocol were approved by the Institutional Review Board. Written informed consent was obtained from each participant.

Data Collection/Clinical Evaluation

Participants completed a self-administered questionnaire such as demographic data and AUDIT-C. The Time Line Follow-Back (TLFB) questionnaire was used to determine the amount of alcohol consumption over the 30-day period before the study date. It was administered in person by trained study coordinators who reviewed the instructions with the subjects prior to administering the questionnaire. The TLFB offers a retrospective report of daily alcohol consumption over the past 30 days; drinks per drinking occasion and pattern of drinking can be computed (Sobell et al., 1988; Vakili et al., 2008). In addition, blood samples were obtained for assay of commonly used markers to identify chronic alcohol use (such as GGT, CDT, AST, ALT, and MCV).

Sample Preparation, Mass Spectrometry, Protein Identification, and Relative Quantification

Seven milliliters of blood were drawn from each subject and centrifuged at 1500×g for 10 minutes at room temperature, within 1-3 hour(s) after acquisition. Aliquots (0.5 mL) were placed in separate 2 mL cryovials and stored at −80° C. until analysis. High-abundance proteins were depleted from each serum sample aliquot as described in the ProteoPrep® 20 User Guide. Briefly, 100 μL of diluted sample (400 μg) was placed on the equilibrated spin column and incubated at room temperature for 20 minutes. The spin column and collection tube were then centrifuged at 2000×g for 30 seconds at room temperature. The flow-through volume was saved in the collection tube. The spin column was then washed twice with 100 μL of equilibration buffer. Total fluid collection was 300 μL. The proteins bound to the column were eluted with 2 mL of elution solution. Each sample was processed as two technical replicates. The two replicates were pooled and concentrated to 300 μL. A final depletion was carried out on the concentrated depleted plasma. These protein samples were subsequently referred to as “depleted samples”. Protein concentration was determined by the Bradford assay1. A 20 μg aliquot of each sample was placed into a new tube and dried via SpeedVac. Depleted serum samples were reconstituted in 50 μL of 4 M urea and then reduced and alkylated using TEP and iodoethanol as described by Lai X, Bacallao R L, Blazer-Yost B L, Hong D, Mason S B, Witzmann F A. Characterization of the renal cyst fluid proteome in autosomal dominant polycystic kidney disease (ADPKD) patients. PROTEOMICS—CLINICAL APPLICATIONS 2008; 2:1140-1152. Briefly, 50 μL of the reduction/alkylation cocktail was added to the protein solution. The sample was incubated at 35° C. for 60 minutes, dried by SpeedVac, and reconstituted with 50 μL of 100 mM NH₄HCO₃ at pH 8.0. A 37.5 μL aliquot of a 20 μg/mL trypsin solution was added to the sample and incubated at 35° C. for 3 hours, after which another 37.5 μL of trypsin was added, and the solution incubated at 35° C. for 3 hours. The digested samples were analyzed using a Thermo-Finnigan linear ion-trap (LTQ) mass spectrometer coupled with a Surveyor autosampler and MS HPLC system (Thermo-Finnigan). Tryptic peptides were injected onto a C18 reversed phase column (TSKgel ODS-100V, 3 μm, 1.0 mm×150 mm) at a flow rate of 50 μL/min. The mobile phases A, B, and C were 0.1% formic acid in water, 50% ACN with 0.1% formic acid in water, and 80% ACN with 0.1% formic acid in water, respectively. The gradient elution profile was as follows: 10% B (90% A) for 7 minutes, 10-67.1% B (90-32.9% A) for 163 minutes, 67.1-100% B (32.9-0% A) for 10 minutes, and 100-50% B (0-50% C) for 10 minutes. The data were collected in the “Data dependent MS/MS” mode with the ESI interface using normalized collision energy of 35%. Dynamic exclusion settings were set to repeat count 1, repeat duration 30 seconds, exclusion duration 120 seconds, and exclusion mass width 0.60 m/z (low) and 1.60 m/z (high).

The acquired data were searched against the UniProt protein sequence database of HUMAN (released on Oct. 31, 2012) using SEQUEST (v. 28 rev. 12) algorithms in Bioworks (v. 3.3). General parameters were set to: mass type set as “monoisotopic precursor and fragments”, enzyme set as “trypsin(KR)”, enzyme limits set as “fully enzymatic—cleaves at both ends”, missed cleavage sites set at 2, peptide tolerance 2.0 amu, fragment ion tolerance 1.0 amu, fixed modification set as +44 Da on Cysteine. The searched peptides and proteins were validated by PeptideProphet³ and ProteinProphet⁴ in the Trans-Proteomic Pipeline (TPP, v. 3.3.0) (http://tools.proteomecenter.org/software.php). Only proteins and peptides with protein probability ≧0.9000 and peptide probability ≧0.8000 were reported. After TPP validation, proteins identified by one peptide were included. Protein quantification was performed using a label-free quantification software package, IDENTIQUANTXL™. Data were considered reliable when the p-value was less than 0.05 and the error factor (EF)<2. The fold changes ratio >1.8 (up regulated) or <0.8 (down regulated) were selected as the cutoff values to designate significant changes in the protein expression.

Enzyme-Linked Immunosorbent Assay

To validate the differential protein level identified by quantitative mass spectrometry approach in the discovery phase, ELISA assay was carried out in the serum sample of subjects in the discovery cohort. Only four proteins were confirmed to be elevated. The following commercially available kits were used to measure the concentrations of proteins in each serum sample of the verification cohort: AT-rich interactive domain-containing protein 4B (Cat#ABIN1122685), Phosphatidylcholine-sterol acyltransferase (antibody-online Cat #ABIN577492), Hepatocyte growth factor-like protein (Cat#ABIN1153476), and ADP-ribosylation factor 6 (Cat#ABIN810899). The assays were performed in duplicates according to the ELISA manufacturer's protocol.).

Statistical Analysis

Basic descriptive statistics, including mean, standard deviations (SD), and percentages, were used to characterize the study subjects. Appropriate comparison tests including chi-square test and Student's t-test were used for comparison between groups for categorical and continuous variables, respectively (FIG. 1). For ELISA results, mean (±SD) of the serum level of the proteins of interest was calculated and used to construct a diagnostic model. For verification cohort, receiver operating characteristic (ROC) curves of each protein levels predicting the excessive alcohol consumption and the area under curve (AUC) were obtained. Sensitivity and specificity analyses of each protein of interest were calculated for the verification cohort. Principal component analysis (PCA) was also performed to identify the most influential bio-marker or the combination with the largest impact on detecting excessive alcohol use. All analyses were conducted with SPSS (Chicago, Ill.).

### Results

Clinical Characteristics of Discovery and Verification Cohorts

The clinical characteristics are presented in Table 1. In both discovery and verification cohorts, there were no statistically significant differences in age, gender, race, and body mass index between controls and excessive drinkers, but 30-day drinking histories were quite different. In the discovery cohort, excessive drinkers had higher AUDIT-C scores (29 vs. 4), greater total standard drinks in the past 30 days (335 vs. 15 drinks), higher average drinks per drinking day (15.2 vs. 2.2 drinks), and a higher number of drinking days in the past month (24 vs. 5 days), when compared to controls. They had significantly higher levels of serum AST, ALT, GGT, % CDT, and MCV. The patterns of alcohol consumption based on TLFB in the verification cohort were similar to those in the discovery cohort with elevated levels of AST, ALT, GGT, % CDT, and MCV in excessive drinkers compared to controls.

TABLE 1
Characteristics of the Discovery and Verification Cohorts

| Discovery Cohort | | | Verification Cohort | | |
|---|---|---|---|---|---|
| | Excessive | | | Excessive | |
| Controls | drinkers | p- | Controls | drinkers | p- |
| (n = 49) | (n = 54) | Value | (n = 40) | (n = 40) | Value |

Demographic and Clinical Characteristics

| | | | | | | |
|---|---|---|---|---|---|---|
| Age (years) | 39.8 ± 9.2 | 44.7 ± 11.4 | 0.10 | 31.4 ± 4.4 | 33.2 ± 4.7 | 0.08 |
| Male sex, n (%) | 43 (88%) | 41 (76%) | 0.12 | 34 (85%) | 33 (82%) | 1.00 |
| Race, n (%) | | | | | | |
| White | 40 (82%) | 45 (83%) | 0.32 | 34 (85%) | 35 (87%) | 0.80 |
| Black | 4 (8%) | 8 (15%) | | 4 (10%) | 4 (10%) | |
| Body mass | 28.8 ± 4.2 | 27.9 ± 4.8 | 0.30 | 30.1 ± 5.1 | 27.9 ± 7.1 | 0.12 |
| index (kg/m²) | | | | | | |
| AUDIT-C | 3.9 ± 3.8 | 29.2 ± 5.9 | 0.0001 | 4.7 ± 5.8 | 28.7 ± 6.9 | 0.001 |

Alcohol drinking patterns during the last 30 days from Time Line Follow-Back

| | | | | | | |
|---|---|---|---|---|---|---|
| Total drinks | 14.5 ± 15.3 | 335.8 ± 141.1 | 0.0001 | 13.2 ± 13.8 | 217.5 ± 137.3 | 0.0001 |
| Number of days | 5.3 ± 4.5 | 23.8 ± 4.8 | 0.0001 | 5.0 ± 5.3 | 19.3 ± 5.8 | 0.0001 |
| drinking last 30 | | | | | | |
| days | | | | | | |
| Average drinks | 2.2 ± 2.2 | 15.2 ± 6.9 | 0.0001 | 2.1 ± 2.2 | 11.0 ± 5.1 | 0.0001 |
| per drinking day | | | | | | |
| Average drinks | 0.4 ± 0.60 | 11.1 ± 4.7 | 0.0001 | 0.4 ± 0.4 | 7.3 ± 4.5 | 0.0001 |
| per day | | | | | | |
| Number of | 0 | 21.4 ± 6.1 | 0.0001 | 0 | 20.3 ± 6.4 | 0.0001 |
| heavy drinking | | | | | | |
| days | | | | | | |
| Greatest number | 2.8 ± 4.3 | 19.8 ± 8.4 | 0.0001 | 3.6 ± 4.1 | 17.4 ± 6.9 | 0.0001 |
| of drinks in 1 | | | | | | |
| day | | | | | | |

Laboratory measures

| | | | | | | |
|---|---|---|---|---|---|---|
| Serum bilirubin | 0.7 ± 1.3 | 0.8 ± 0.6 | 0.40 | 0.6 ± 0.4 | 0.6 ± 0.3 | 0.46 |
| (mg/dl) | | | | | | |
| Serum aspartate | 23.5 ± 10.6 | 31.7 ± 17.1 | 0.007 | 23.5 ± 11.1 | 31.3 ± 16.6 | 0.02 |
| aminotransferase | | | | | | |
| (U/l) | | | | | | |
| Serum alanine | 32.8 ± 21.0 | 45.8 ± 19.6 | 0.002 | 30.3 ± 10.1 | 37.6 ± 16.8 | 0.02 |
| aminotransferase | | | | | | |
| (U/l) | | | | | | |
| Serum albumin | 4.2 + 0.4 | 3.7 + 0.4 | 0.001 | 4.2 + 0.3 | 3.6 + 0.3 | 0.001 |
| (g/dl) | | | | | | |
| Serum gamma- | 31.5 + 16.7 | 44.9 + 13.9 | 0.04 | 43.6 + 53.7 | 82.8 + 90.0 | 0.03 |
| glutamyl | | | | | | |
| transpeptidase | | | | | | |
| (U/l) | | | | | | |
| Serum | 1.4 ± 0.8 | 2.9 ± 1.4 | 0.001 | 1.9 ± 0.6 | 2.3 ± 1.0 | 0.009 |
| percentage of | | | | | | |
| carbohydrate- | | | | | | |
| deficient | | | | | | |
| transferrin | | | | | | |
| (% CDT, %) | | | | | | |
| Mean | 89.9 ± 3.9 | 93.8 ± 6.4 | 0.001 | 89.4 ± 5.6 | 93.3 ± 5.8 | 0.005 |
| corpuscular | | | | | | |
| volume (fl) | | | | | | |

Protein Identification

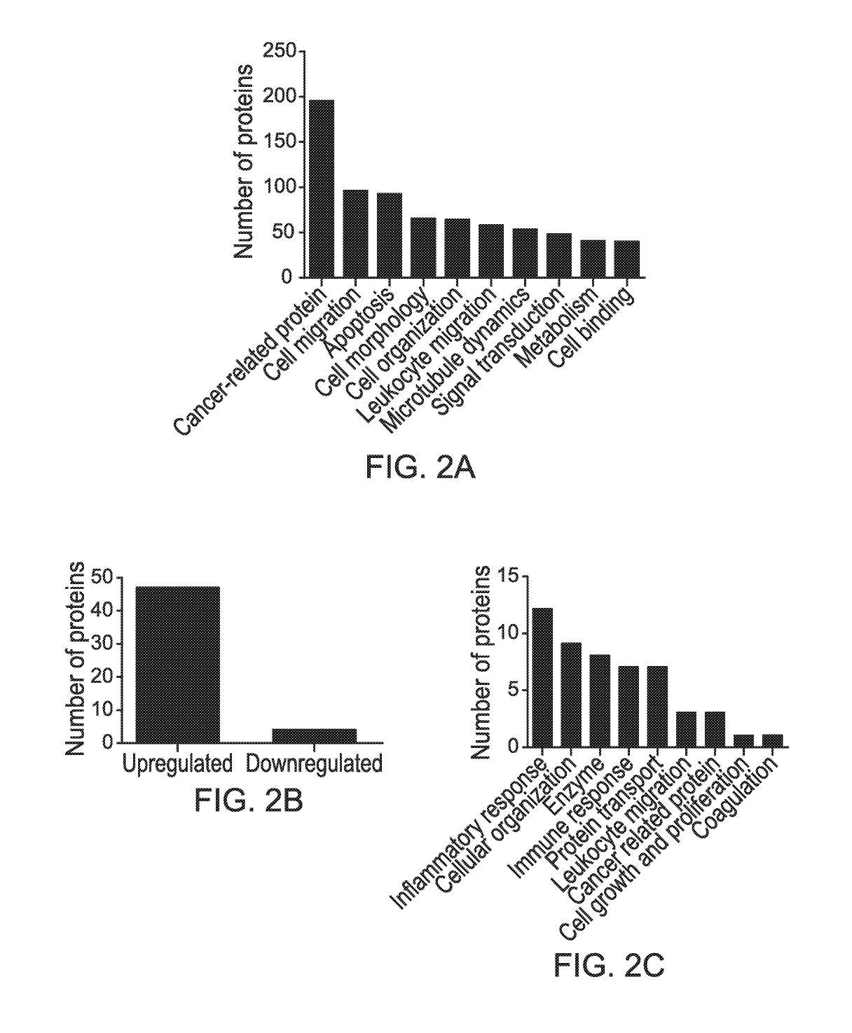

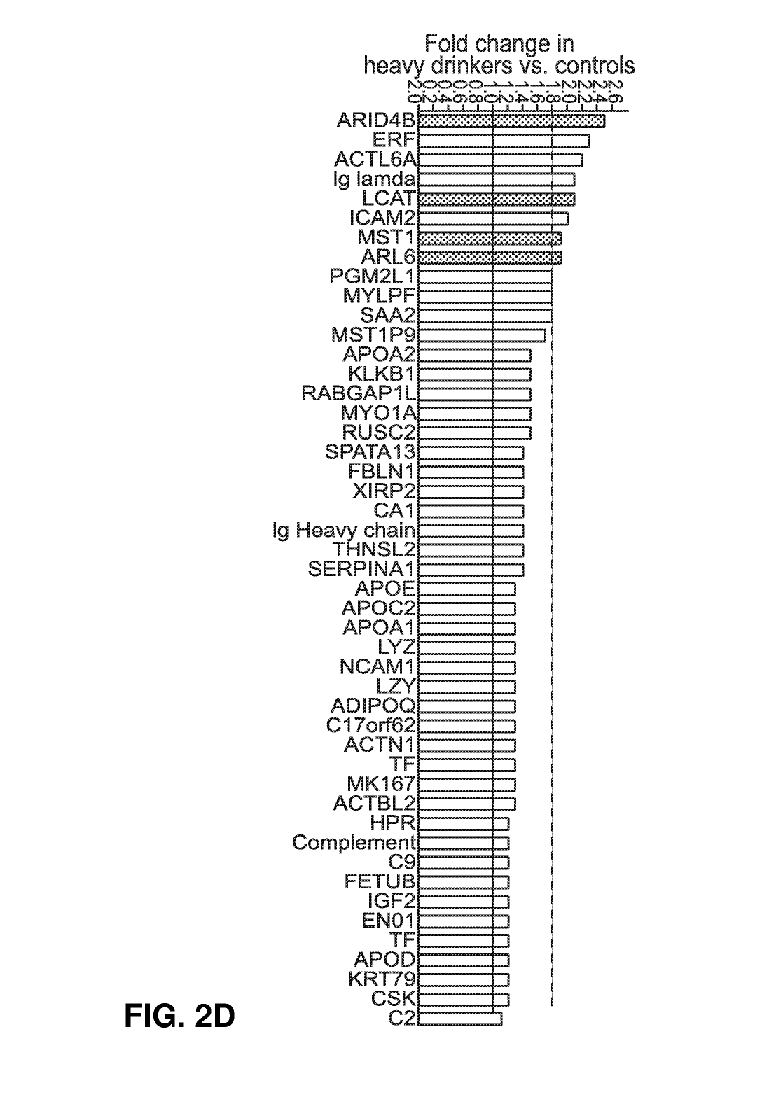

In the discovery cohort, proteomic analysis identified and quantified 602 unique proteins. As shown in FIG. 2A, these proteins were involved in several cellular functions. Of these 602 proteins, 51 proteins had the potential to separate subjects with excessive alcohol use from controls and 47 were elevated in excessive drinkers (using cutoff p-value at 0.01; FIGS. 2B-2D). The informative proteins were involved in inflammatory responses, cellular organization, enzymes, and immune responses (FIG. 2C).

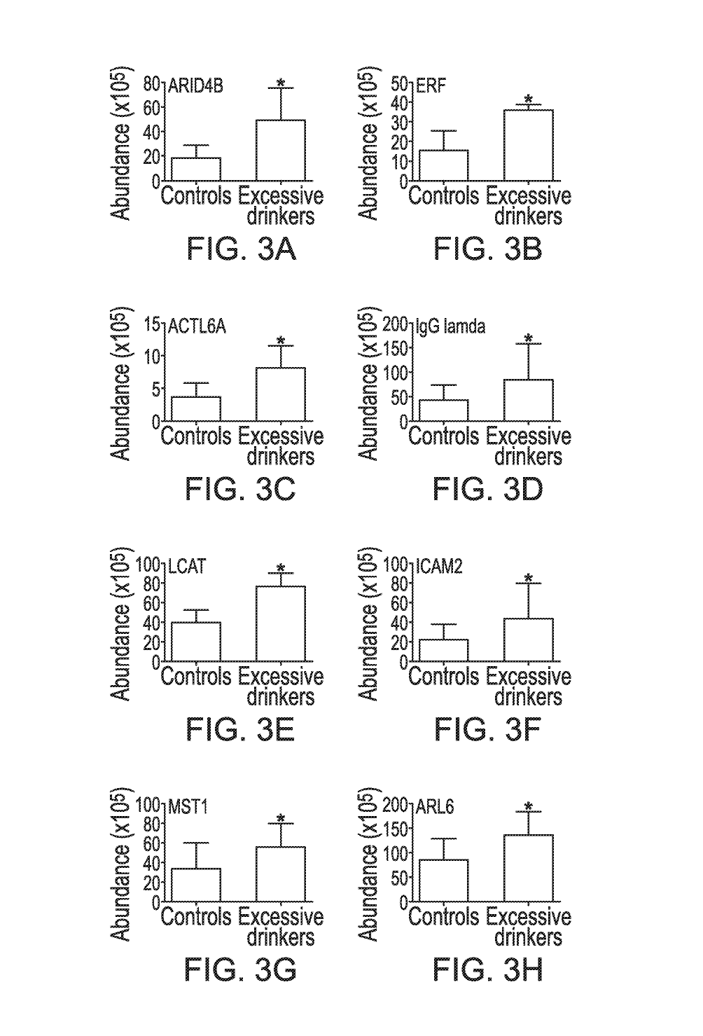

Next, the ratio of >1.8 comparing the detected levels of protein between groups in the discovery cohort, was determined as the cutoff value to designate significant differences in the protein expression, and 8 proteins were found that met this criterion: AT-rich interactive domain-containing protein 4B (ARID4B), ETS domain-containing transcription factor ERF, actin-like protein 6A, immunoglobulin lambda, phosphatidylcholine-sterol acyltransferase (LCAT), intercellular adhesion molecule 2, hepatocyte growth factor-like protein (MST1), and ADP-ribosylation factor 6 (ARL6) (FIGS. 2D and 3). To confirm the findings from the LC/MS, the confirmatory ELISA assays were performed in the serum samples of subjects in the discovery cohort, and it was found that the serum levels of the following 4 proteins were significantly elevated in the serum of excessive drinkers relative to controls: ARID4B, LCAT, MST1, and ARL6.

Verifications of the Proteins as the Candidates for Biomarker

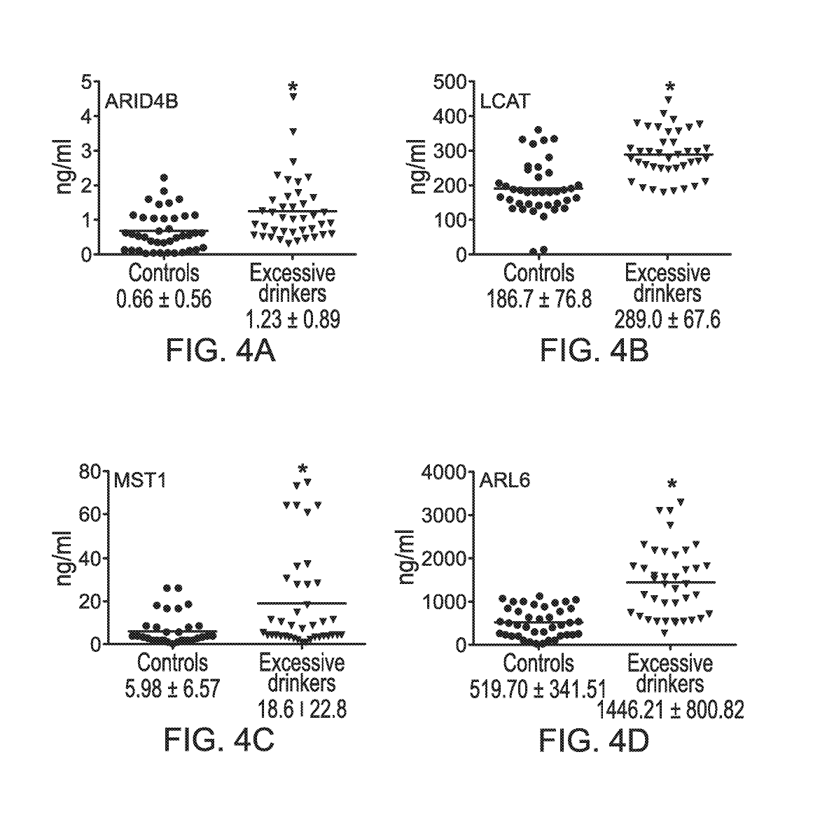

To validate the results from the discovery cohort, the serum levels of ARID4B, LCAT, MST1, and ARL6 were compared between the 2 groups in the verification cohort. The results of the ELISA for these 4 proteins are shown in FIG. 4 and Table 2; the serum levels of these 4 proteins were significantly higher in excessive drinkers compared to controls, confirming the results from the discovery cohort.

TABLE 2

| | | ELISA | LC-MS | |
|---|---|---|---|---|
| | Gene | fold | fold | ELISA |
| Protein | Symbol | difference | difference | p-value |
| AT-rich interactive domain- | ARID4B | 1.87 | 2.5 | 0.0009 |
| containing protein 4B | | | | |
| Phosphatidylcholine-sterol | LCAT | 1.55 | 2.1 | 0.0004 |
| acyltransferase | | | | |
| Hepatocyte growth factor- | MST1 | 3.1 | 1.9 | 0.0001 |
| like protein | | | | |
| ADP-ribosylation factor 6 | ARL6 | 2.78 | 1.9 | 0.0002 |

Performance of the Proteins in the Differential Diagnosis of Excessive Drinkers from Controls in Comparison to the Conventional Markers

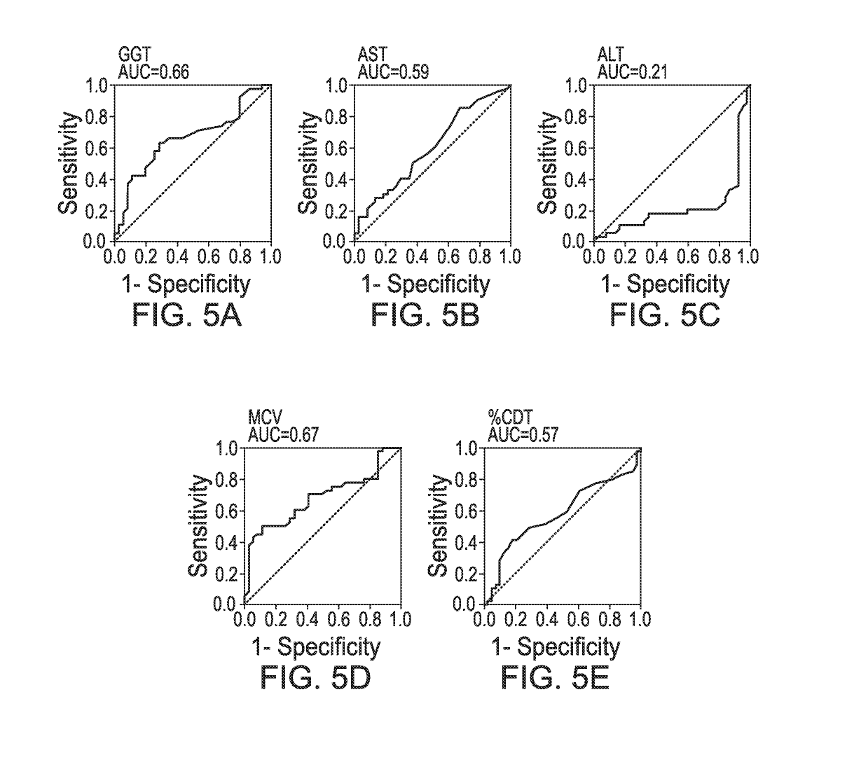

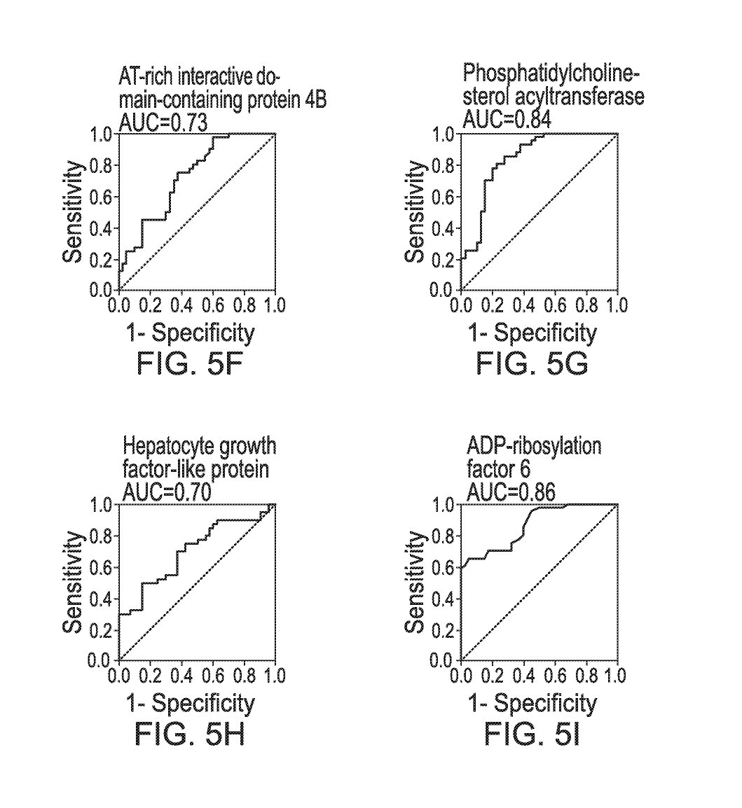

The ROC, AUC values, sensitivity, and specificity of the new novel proteins in the verification cohort appear in Table 3 and are compared to those indices for the conventional markers commonly used to screen for excessive alcohol use. FIGS. 5A-5I show the ROC curves for these markers. The ROC of the conventional markers discriminating between excessive alcohol use and controls had an AUC ranging from 0.21 (for serum ALT) to 0.67 (for MCV). Using the thresholds reported in Table 3, the conventional tests were diagnostic of excessive alcohol use with 76% sensitivity and 71% specificity (for GGT), 85% sensitivity and 67% specificity (for AST), 75% sensitivity and 91% specificity (for ALT), 85% sensitivity and 85% specificity (for MCV), and 82% sensitivity and 86% specificity (for % CDT). In comparison, the new markers demonstrated superior discrimination to distinguish excessive drinkers from controls when compared to conventional markers. The sensitivity was 97% (for ARID4B and ARL6), 95% (for LCAT), and 90% (for MST1) (Table 3). The ROC curves of these proteins showed the improvement in the detection of excessive drinkers compared to conventional laboratory tests (Table 3 and FIGS. 5A-5I, p<0.05 for the AUC of the new markers compared to each of the conventional marker). The AUC ranged from 0.73 (for ARID4B) to 0.86 (for ARL6).

TABLE 3
Performance of the Proteins in the Differential Diagnosis of
Excessive Drinkers from Controls in Comparison to the
Conventional Markers

| | | | | Area under |
|---|---|---|---|---|
| | | Sensi- | Speci- | the Curve |
| Test | Cutoff | tivity | ficity | (95% CI) |
| Gamma-glutamyl | 23 | 0.76 | 0.71 | 0.66 (0.53-0.78) |
| transpeptidase | | | | |
| Aspartate | 17.5 | 0.85 | 0.67 | 0.59 (0.46-0.72) |
| aminotransferase | | | | |
| Alanine aminotransferase | 16.5 | 0.75 | 0.91 | 0.21 (0.10-0.32) |
| Mean corpuscular | 86.5 | 0.85 | 0.85 | 0.67 (0.55-0.80) |
| volume | | | | |
| Percentage of | 1.45 | 0.82 | 0.86 | 0.57 (0.44-0.70) |
| carbohydrate-deficient | | | | |
| transferrin | | | | |
| AT-rich interactive | 0.32 | 0.97 | 0.70 | 0.73 (0.62-0.83) |
| domain-containing | | | | |
| protein 4B | | | | |
| Phosphatidylcholine- | 184.1 | 0.95 | 0.47 | 0.84 (0.75-0.93) |
| sterol acyltransferase | | | | |
| Hepatocyte growth | 2.07 | 0.90 | 0.72 | 0.70 (0.58-0.81) |
| factor-like protein | | | | |
| ADP-ribosylation | 283.3 | 0.97 | 0.65 | 0.86 (0.78-0.94) |
| factor 6 | | | | |

PCA was carried out to identify potential linear combination of the conventional and the new markers that accurately predict excessive alcohol consumption using the verification cohort. As shown in Tables 3 and 4, the first PCA accounted for ˜97.8% of the variation of the data set. PCA also showed the levels of ARL6 had the loading for the first principal component of 0.998 (Table 5), while the loading for other markers was <0.05. This data indicated that ARL6 had the best ability to discern subjects with excessive alcohol use, when using alone and compared to other conventional/new markers.

TABLE 4
The Principal Component Analysis to Determine the
Ability of each marker or in Combination to Detect
Subjects with Excessive Alcohol Use. Proportion of
Variance Explained by the First 3 Principal Components
Eigenvalues of the covariance matrix

| | | | Cumulative |
|---|---|---|---|
| Principal | | Proportion of | proportion of |
| component | Eigenvalue | Variance | Variance |
| 1 | 645515.565 | 0.978 | 0.978 |
| 2 | 7722.426 | 0.012 | 0.990 |
| 3 | 5110.214 | 0.008 | 0.998 |

TABLE 5
The Principal Component Analysis to Determining the
Ability of Each Marker or in Combination to Detect
Subjects Use: Factor Loading for the First 3 Components

| | Principal | Principal | Principal |
|---|---|---|---|
| Markers | Component 1 | Component 2 | Component 3 |
| Aspartate | 0.005 | 0.184 | 0.131 |
| aminotransferase | | | |
| Alanine | −0.008 | 0.156 | 0.202 |
| aminotransferase | | | |
| Mean corpuscular | 0.002 | 0.013 | −0.004 |
| volume | | | |
| Percentage of | 0.000 | −0.003 | −0.001 |
| carbohydrate-deficient | | | |
| transferrin | | | |
| Gamma-glutamyl | 0.011 | 0.731 | 0.594 |
| transpeptidase | | | |
| Phosphatidylcholine- | 0.049 | 0.637 | −0.766 |
| sterol acyltransferase | | | |
| AT-rich interactive | 0.000 | 0.000 | 0.001 |
| domain-containing | | | |
| protein 4B | | | |
| Hepatocyte growth | 0.008 | 0.006 | −0.030 |
| factor-like protein | | | |
| ADP-ribosylation | 0.998 | −0.039 | 0.032 |
| factor 6 | | | |

Correlations Between the Levels of Conventional Laboratory Tests/the New Markers and the Level of Alcohol Consumption During the Past 30 Days

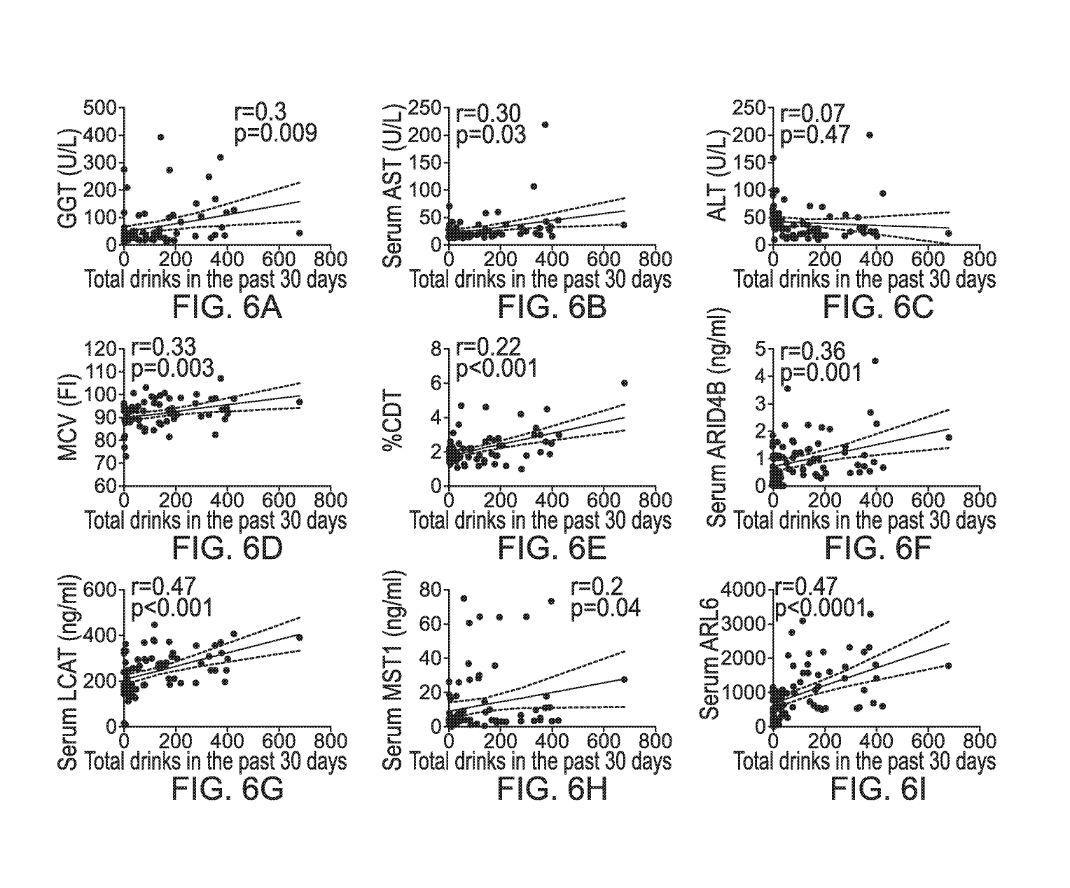

The correlations were computed between the levels of the conventional enzyme assays and the levels of alcohol consumption measured by the TLFB during the last 30 days for the verification cohort (FIGS. 6A-6I): GGT (r=0.3), AST (r=0.3), MCV (r=0.3), and % CDT (r=0.2), respectively. The levels of ALT, on the other hand, had no significant correlation with the intensity of recent alcohol consumption in the last 30 days as measured by TLFB. The same correlations were computed for the new markers; these were comparable to the conventional assays, with the notable exception that LCAT and ARL6 had r=0.47 (p<0.001 and p<0.0001, respectively), accounting for twice the variance in the data set as did the best conventional marker, MCV.

The above data shows significantly increased serum expression of 4 proteins in excessive drinkers, which was confirmed by consistent findings in the verification cohort. The newly identified proteins performed better (according to the AUC of the ROC results) than the conventional laboratory tests routinely used to screen for excessive alcohol use. The AUC for the new markers ranged from 0.73 to 0.86 compared to that from 0.21 to 0.67 (for conventional markers).

### Example 2

In this Example, levels of serum sCD14 and sCD163 were evaluated as markers of excessive alcohol use.

In this Example, 102 subjects were analyzed using the evaluation methods as described in Example 1 above. The detailed characteristics showing the levels of conventional makers for excessive alcohol use (EAU) as well as sCD14 and sCD163 are shown in Table 6.

TABLE 6
Demographic and clinical characteristics of study subjects

| Variables | Controls (n = 51) | EAU (n = 51) | P-value |
|---|---|---|---|
| Age (Yrs) | 32 ± 9 | 39 ± 13 | 0.01 |
| Male (%) | 69% | 67% | 0.68 |
| Race (White, %) | 49% | 50% | 0.19 |
| AUDIT scores | 4.2 ± 4.0 | 26.6 ± 8.2 | 0.001 |
| Total drinks in 30 | 15 ± 15 | 204 ± 121 | 0.0001 |
| days, drinks | | | |
| AST, U/L | 23 ± 10 | 35 ± 25 | 0.01 |
| ALT, U/L | 49 ± 18 | 35 ± 33 | 0.01 |
| MCV,fL | 90 ± 3.9 | 92.5 ± 6.1 | 0.03 |
| % CDT | 1.5 ± 0.8 | 2.4 ± 1.3 | 0.01 |
| GGT, U/L | 35 ± 27 | 68 ± 68 | 0.01 |
| LPS (EU/ml) | 1.7 ± 0.77 | 4.8 ± 1.2 | 0.001 |
| sCD14 (ng/ml) | 1,266 ± 448 | 3,353 ± 1444 | 0.001 |
| sCD163 (pg/ml) | 85 ± 8.7 | 396.9 ± 171 | 0.001 |

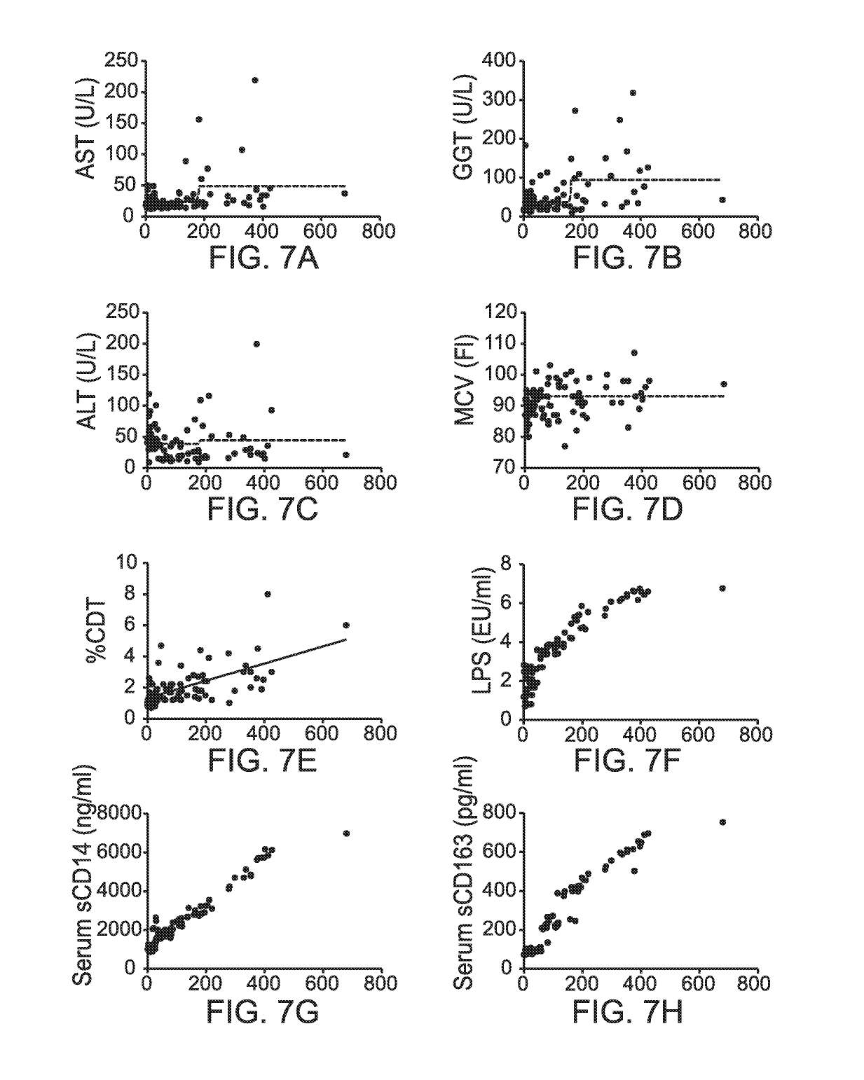

The levels of conventional markers, except for ALT, were elevated in EAU subjects. These findings are compatible with previous reports showing that AST is more sensitive to screen for EAU than ALT1. Interestingly, however, it was also found that the levels of serum sCD14 and sCD163 were significantly elevated in the serum of EAU subjects. Further, there was found to be a strong correlation between the levels of sCD14 and sCD163 and the total drinks in the past 30 days and the lack of such correlations when the analyses were performed with conventional markers (FIGS. 7A-7H).

### Example 3

In this Example, the level of serum sCD40 was evaluated as a marker of excessive alcohol use.

In this Example, 74 subjects were analyzed using the evaluation methods as described in Example 1 above. The detailed characteristics showing the levels of conventional makers for excessive alcohol use (EAU) as well as sCD40 are shown in Table 7.

TABLE 7
Demographic and clinical characteristics of study subjects

| Variables | Controls (n = 30) | EAU (n = 44) | P-value |
|---|---|---|---|
| AUDIT scores | 3.7 ± 3.2 | 27.7 ± 7.5 | 0.001 |
| Total drinks in 30 | 13 ± 15 | 210 ± 117 | 0.0001 |
| days, drinks | | | |
| AST, U/L | 25 ± 12 | 36 ± 38 | 0.01 |
| ALT, U/L | 33 ± 33 | 55 ± 22 | 0.01 |
| MCV, fL | 89 ± 4.0 | 92 ± 6 | 0.03 |
| % CDT | 1.2 ± 0.2 | 2.5 ± 1.4 | 0.01 |
| GGT, U/L | 36 ± 32 | 64 ± 81 | 0.01 |
| sCD40 (pg/ml) | 474.9 ± 258 | 915.4 ± 379 | 0.001 |

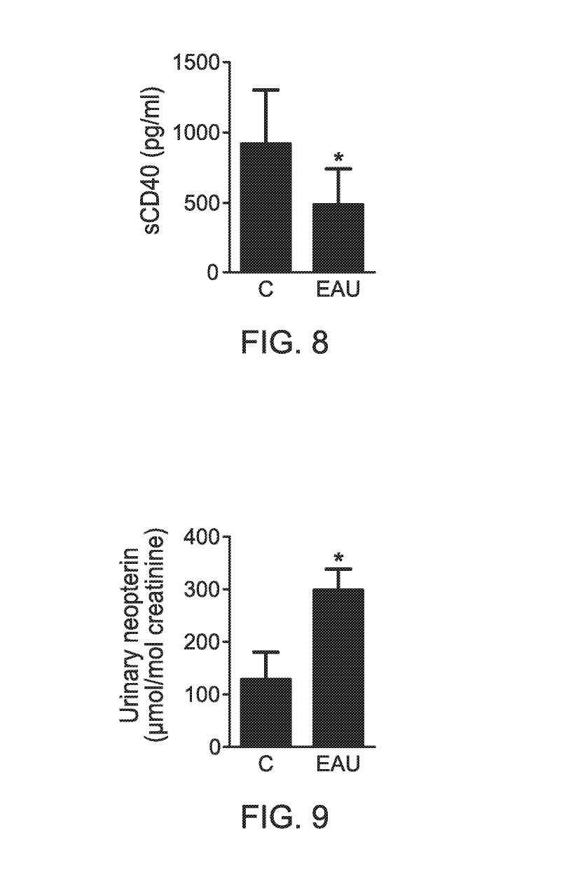

As shown in FIG. 8, in a cohort of 74 subjects, the levels of sCD40 were significantly lower in subjects with EAU compared to those in controls.

### Example 4

In this Example, the level of urine neopterin was evaluated as a marker of excessive alcohol use.

In this Example, 39 subjects were analyzed using the evaluation methods as described in Example 1 above. Urinary levels of neopterin were measured in 18 control subjects and 21 subjects previously diagnosed with excessive alcohol use (EAU). As shown in FIG. 9, a significant increase in urinary neopterin in subjects with EAU was seen as compared to controls.

In summary, diagnostic proteins have been identified from serum that demonstrate the ability to discern subjects with recent, excessive alcohol use from controls.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A method for diagnosing excessive alcohol use in a subject, the method comprising:
obtaining a sample from a subject suspected of excessive alcohol use;
contacting the sample with an agent that specifically binds to a biomarker selected from the group consisting of AT-Rich Interactive Domain-Containing Protein 4B (ARID4B), phosphatidylcholine-sterol acyltransferase (LCAT), hepatocyte growth factor-like protein (MST1), ADP-ribosylation factor 6 (ARL6), sCD14, sCD163, and neopterin, and combinations thereof, to form a complex between the agent and biomarker;
detecting the complex to determine an expression level of the biomarker in the sample; and
diagnosing excessive alcohol use in the subject if the expression level of the biomarker is increased as compared to the expression level of the biomarker in a reference sample.

2. The method of claim 1, wherein the sample is selected from the group consisting of serum, plasma, whole blood and urine.

3. The method of claim 1, wherein the biomarker is AT-Rich Interactive Domain-Containing Protein 4B (ARID4B).

4. The method of claim 1, wherein the biomarker is ADP-ribosylation factor 6 (ARL6).

5. The method of claim 1, wherein the biomarkers are AT-Rich Interactive Domain-Containing Protein 4B (ARID4B), phosphatidylcholine-sterol acyltransferase (LCAT), hepatocyte growth factor-like protein (MST1), and ADP-ribosylation factor 6 (ARL6).

6. The method of claim 1, wherein the biomarkers are sCD14 and sCD163.

7. The method of claim 1, wherein the agent is selected from an antibody, a ligand, and combinations thereof.

8. The method of claim 1 further comprising completing a self-administered questionnaire by the subject at risk for excessive alcohol use.

9. A method for diagnosing excessive alcohol use in a subject, the method comprising:
obtaining a sample from a subject suspected of excessive alcohol use;
analyzing the sample by liquid chromatography/mass spectrometry to determine a concentration of a biomarker selected from the group consisting of AT-Rich Interactive Domain-Containing Protein 4B (ARID4B), phosphatidylcholine-sterol acyltransferase (LCAT), hepatocyte growth factor-like protein (MST1), ADP-ribosylation factor 6 (ARL6), sCD14, sCD163, and neopterin, and combinations thereof; and
diagnosing excessive alcohol use in the subject if the concentration level of the biomarker is increased as compared to the concentration of the biomarker in a reference sample.

10. The method of claim 9, wherein the sample is selected from the group consisting of serum, plasma, whole blood and urine.

11. The method of claim 9, wherein the biomarker is AT-Rich Interactive Domain-Containing Protein 4B (ARID4B).

12. The method of claim 9, wherein the biomarker is ADP-ribosylation factor 6 (ARL6).

13. The method of claim 9, wherein the biomarkers are AT-Rich Interactive Domain-Containing Protein 4B (ARID4B), phosphatidylcholine-sterol acyltransferase (LCAT), hepatocyte growth factor-like protein (MST1), and ADP-ribosylation factor 6 (ARL6).

14. The method of claim 9, wherein the biomarkers are sCD14 and sCD163.

15. The method of claim 9 further comprising completing a self-administered questionnaire by the subject at risk for excessive alcohol use.

16. A method for diagnosing excessive alcohol use in a subject, the method comprising:
obtaining a sample from a subject suspected of excessive alcohol use;
contacting the sample with an agent that specifically binds to sCD40, to form a complex between the agent and sCD40;
detecting the complex to determine an expression level of sCD40 in the sample; and
diagnosing excessive alcohol use in the subject if the expression level of the sCD40 is decreased as compared to the expression level of the sCD40 in a reference sample.

17. The method of claim 16, wherein the agent is selected from an antibody, a ligand, and combinations thereof.

18. The method of claim 1 further comprising completing a self-administered questionnaire by the subject at risk for excessive alcohol use.

19. A biomarker panel for diagnosing excessive alcohol use, the biomarker panel comprising at least two biomarkers selected from the group consisting of AT-rich interactive domain-containing protein 4B (ARID4B), ETS domain-containing transcription factor ERF (ERF), actin-like protein 6A (ACTL6A), IgG (immunoglobulin lambda), phosphatidylcholine-sterol acyltransferase (LCAT), intercellular adhesion molecule 2 (ICAM2), hepatocyte growth factor-like protein (MST1), and ADP-ribosylation factor 6 (ARL6), sCD14, sCD163, and sCD40.

20. The biomarker panel of claim 19, wherein the biomarkers are AT-Rich Interactive Domain-Containing Protein 4B (ARID4B), phosphatidylcholine-sterol acyltransferase (LCAT), hepatocyte growth factor-like protein (MST1), and ADP-ribosylation factor 6 (ARL6).
